(19) United States
(12) Patent Application Publication
(10) Pub. No.: US 2010/0120723 A1
(43) Pub. Date: May 13, 2010

(54) PHARMACEUTICAL COMPOSITION COMPRISING A HOT-MELT GRANULATED LUBRICANT

(76) Inventors: Mostafa Akbarieh, Richmond Hill (CA); Thinesh Sivarajah, Scarborough (CA); Shetal Shah, Toronto (CA)

Correspondence Address: GREENLEE WINNER AND SULLIVAN P C, 4875 PEARL EAST CIRCLE, SUITE 200, BOULDER, CO, 80301, US

(21) Appl. No.: 12/519,341

(22) PCT Filed: Dec. 19, 2007
(86) PCT No.: PCT/CA07/02314

§ 371 Date: Jan. 13, 2010

(30) Foreign Application Priority Data
Dec. 20, 2006 (CA) 2571559

(51) Int. Cl. A61K 47/38; A61K 47/30; A61K 47/36; A61K 47/42; A61K 47/02; A61K 31/663; A61P 19/08; A61P 19/10

(52) U.S. Cl. 514/108; 514/774; 514/781; 514/779; 514/772; 514/782; 514/777; 514/770; 514/773; 514/784; 514/778

(57) ABSTRACT

The present invention relates to a lubricant granulate prepared using a hot melt granulation process, or thermal-heat process. The lubricant granulate is useful in facilitating the use of higher concentrations of lubricant than typically possible in pharmaceutical compositions. Also provided are pharmaceutical compositions comprising the lubricant granulate. Such pharmaceutical compositions can contain bisphosphonic acid as the active ingredient and can be suitable for oral administration. The present invention also provides a hot melt process for preparing the lubricant granulate for subsequent use in pharmaceutical compositions.

### CROSS REFERENCE TO RELATED APPLICATION

This application claims the benefit of co-pending Canadian patent application No. 2,571,559, filed Dec. 19, 2006, which are incorporated herein in their entirety.

### FIELD OF THE INVENTION

The present invention pertains to the field of pharmaceutical compositions and methods of preparation thereof and, more particularly, to pharmaceutical compositions comprising a hot-melt granulated lubricant, and to a method of preparation thereof.

### BACKGROUND OF THE INVENTION

Bisphosphonic acid or a pharmaceutically acceptable salt thereof are important in the treatment of bone diseases as well as problems with calcium metabolism such as osteoporosis, Paget's disease, and hypercalcaemia. Bisphosphonates, i.e. bisphosphonic acids or soluble, pharmaceutically acceptable salts thereof, are synthetic analogs of the naturally occurring pyrophosphate. Due to their marked affinity for solid-phase calcium phosphate, Bisphosphonic acid or a pharmaceutically acceptable salt thereof bind strongly to bone mineral. Pharmacologically active Bisphosphonic acid or a pharmaceutically acceptable salt thereof are well known in the art and are potent inhibitors of bone resorption and are therefore useful in the treatment and prevention of diseases involving abnormal bone resorption, such as hypercalcaemia, osteoporosis, tumour osteolysis, Paget's disease, etc. Bones serve as support structures but are also involved in various body stimuli signaling and response mechanisms. Therefore simple prosthetics, made to support the damaged bone, do not give a patient the proper active effect that repairing the bone itself would do.

Bisphosphonic acid or a pharmaceutically acceptable salt thereof as pharmaceutical agents are described for example in U.S. Pat. No. 4,687,767, U.S. Pat. No. 4,666,895, U.S. Pat. No. 4,927,814, U.S. Pat. No. 4,942,157, and U.S. Pat. No. 4,777,163. Pharmaceutical forms of marketed Bisphosphonic acid or a pharmaceutically acceptable salt thereof are either oral formulation (tablets or capsules) or solutions for intravenous injection or infusion. Bisphosphonic acid or a pharmaceutically acceptable salt thereof can be classified into two groups with different modes of action. Sodium ibandronate belongs to the more potent nitrogen-containing Bisphosphonic acid or a pharmaceutically acceptable salt thereof (Russel R. G. G., Rogers M. J. Bisphosphonic acid or a pharmaceutically acceptable salt thereof: From the laboratory to the clinic and back again. Bone 25(1):97-106 (1999). Sodium ibandronate is one of the most potent Bisphosphonic acid or a pharmaceutically acceptable salt thereof currently marketed in osteoporosis (BONIVA) and metastatic bone diseases. Sodium ibandronate, in animal models of bone resorption, has been shown to be many times more potent than alendronate, risedronate, pamidronate and clodronate (Muhlbauer R. C., Bauss R., Schenk R., Janner M., Bosies E., Strein K., and Fleisch H. BM21.0955 A Potent New Bisphosphonic acid or a pharmaceutically acceptable salt thereof to Inhibit Bone Resorption. J. Bone Miner. Res. 6:1003-1011 (1991)).

Sodium ibandronate inhibits bone resorption without any affecting mineralization. It has also been shown to decrease osteoclastic activity thus inhibiting bone destruction, since at high doses it reduces the number of osteoclasts.

Pharmaceutical preparations of Bisphosphonic acid or a pharmaceutically acceptable salt thereof, especially sodium ibandronate can be problematic because they inherently cause heavy compression problems during processing. Problems such as increased stickiness or reduced hardness issues leading to undue friability limitations need to be overcome during the preparation of this family of compounds with the use of proper lubricants in the compositions. Normally, the lubricant makes up 0.25-1.5% of the composition and ideally it is kept below 1%.

When using Stearic Acid as the lubricant for other Bisphosphonic acid or a pharmaceutically acceptable salt thereof, it has been noted that greater concentrations are required to properly lubricate the composition but no more than roughly 6% proportion by weight can be used before this particular lubricant no longer contributes to solving the inherent problems associated with compression of this preparation (WO 93/09785 Proctor and Gamble. In U.S. Pat. No. 6,294,196 (Hoffmann-La Roche) the solid pharmaceutical form disclosed contains a lubricant in the form of less than 5% by weight of the stearic acid relative to the total weight of the form of administration. US '196 states that the resulting granulate may become so water-repellent that the resulting drug disintegrates very slowly when stearic acid levels are too high, for example above 5% of the total weight.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide a pharmaceutical composition comprising a hot-melt granulated lubricant. In accordance with one aspect of the present invention there is provided a process for preparing a lubricant granulate comprising: (a) preparing a molten mixture comprising a lubricant and a hot melt binder; (b) allowing the molten mixture to cool; and (c) milling the cooled mixture to form a granulate.

In accordance with another aspect of the present invention there is provided a lubricant granulate that is prepared by a process comprising: (a) preparing a molten mixture comprising a lubricant and a hot melt binder; (b) allowing the molten mixture to cool; and (c) milling the cooled mixture to form a granulate. According to one embodiment of the present invention, the lubricant granulate comprises a lubricant at an amount of at least 35% by weight of the granulate. Preferably, the lubricant is present at an amount of at least 45% by weight.

In accordance with another aspect of the present invention, there is provided a pharmaceutical composition that comprises an active pharmaceutical ingredient (API) and a lubricant granulate prepared by a process comprising: (a) preparing a molten mixture comprising a lubricant and a hot melt binder; (b) allowing the molten mixture to cool; and (c) milling the cooled mixture to form a granulate. In accordance with one embodiment of the present invention, the API is a bisphosphonic acid or a pharmaceutically acceptable derivative thereof, such as ibandronic acid or sodium ibandronate.

In accordance with another aspect of the present invention, there is provided a method of treating a disorder associated with increased bone resorption comprising administering to a patient the pharmaceutical composition which contains the lubricant granulate of the present invention and an active pharmaceutical ingredient, preferably a Bisphosphonic acid or a pharmaceutically acceptable salt thereof and, more preferably, sodium ibandronate. The bone resorption disorder can be, but is not limited to, osteoporosis, hypercalcaemia, tumour osteolysis or Paget's disease.

One advantage of the present invention is that the hot-melt granulated lubricant can make up greater than 5% of the total solid oral dosage, preferably greater than 9%, and more preferably greater than 15%. The lubricant can be chosen from either talc, magnesium stearate, calcium stearate, polyethylene glycol and hydrogenated vegetable oils but it is most preferable to use is stearic acid. The increased lubricant content decreases the stickiness of the preparation and, thus, overcomes the heavy compression problems inherently found with some API's, including the bisphosphonic acids and pharmaceutically acceptable derivatives thereof.

Another advantage of the compositions of the present invention is that hardness or DT levels of the compositions are lowered, thus reducing friability issues during processing, preventing the composition from crumbling away or not ejecting properly.

Overall, the compositions of the present invention do not show a retarded dissolution profile as would be expected with compositions containing similarly high concentrations of stearic acid that has not been prepared by the hot-melt granulation process of this invention.

### DETAILED DESCRIPTION OF THE INVENTION

Surprisingly, the current inventors have found that pharmaceutical compositions can be created comprising an active pharmaceutical ingredient (API) together with high concentrations of lubricant, as long as the lubricant has been prepared using a hot-melt granulation process. The problems of prior art discussed above are solved by creating pharmaceutical compositions that comprise a hot-melt granulated lubricant, which allows for an increased lubrication of a compression challenged composition. The amount of coated lubricant can now increase to values greater than 6% of the entire formulation. Without wishing to be bound by theory, it is predicted that the higher concentrations of lubricant are possible because the hot-melt granulated lubricant avoids intimate contact with the API.

The process of the present invention includes the basic steps of preparing a molten mixture comprising a lubricant and a hot melt binder; allowing the molten mixture to cool; and milling the cooled mixture to form a granulate.

In order to carry out the melt granulation process of the invention, a melt granulating medium containing the lubricant is heated to a temperature at which the medium is at least partially in a molten state. As the granulating medium is heated and becomes molten, it forms liquid bridges between the particles of the composition which change to solid bonds upon cooling. A solid mass is thereby formed in which the granulating medium and the remaining components of the composition are closely bound together, forming agglomerations or granules. The term “melt association” employed herein refers to the bonding relationship among the components of the compositions of the invention which results from the cooling of a molten granulating component in a melt granulation process.

The lubricant can be pre-heated to at least a softened state, and then combined with the hot melt binder, and other components if present, and heat can be continued to be supplied to the resulting composition, as necessary, to carry out the melt granulation. Alternatively, the lubricant is added to pre-heated hot melt binder, and heat is continued to be supplied to the resulting composition, as necessary, to carry out the melt granulation. In this alternative, if additional components are added they are typically added to the molten mixture of the lubricant and hot melt binder.

It will be within the skill of the worker in the art to devise techniques for ordering the heating and mixing of the various constituents of the lubricant granulates of the invention to result in the formation of a suitable melt granulation.

The compositions are maintained at an elevated temperature for a time sufficient to substantially completely liquify the composition of the invention. The length of time that the molten mixture is maintained at an elevated temperature can vary widely. The time will be such that degradation of the components of the mixture will be minimized. The appropriate time can be readily determined experimentally and will vary according to the particular mixture being granulated. Generally, the time of exposure of the components to the elevated temperature is less than 5 minutes and preferably less than 2 minutes.

The resulting composition is then cooled or allowed to cool, preferably to room temperature. If necessary, the composition is also dried (possibly in an oven) to remove water from the mixture prior to granulation.

The cooled mixture is then broken into small chunks and milled using standard procedures. The resulting granulate is suitable for use in the preparation of pharmaceutical formulations.

Lubricant, as used herein, means a material that can reduce the friction arising at the interface of the tablet and the die wall during compression and ejection thereof. Lubricants may also serve to prevent sticking to the punch and, to a lesser extent, the die wall as well. The term “antiadherents” is sometimes used to refer specifically to substances which function during ejection. As used in the present disclosure, however, the term “lubricant” is used generically and includes “antiadherents”. Tablet sticking during formation and/or ejection may pose serious production problems such as reduced efficiency, irregularly formed tablets, and non-uniform distribution of intended agents or ingredients to be delivered thereby. These problems are particularly severe with high speed tableting approaches and methods.

Lubricants may be intrinsic or extrinsic. A lubricant that is directly applied to the tableting tool surface in the form of a film, as by spraying onto the die cavity and/or punch surfaces, is known as an extrinsic lubricant. Although extrinsic lubricants can provide effective lubrication, their use requires complex application equipment and methods that add cost and reduce productivity.

Intrinsic lubricants are incorporated in the material to be tableted. Magnesium, calcium and zinc salts of stearic acid have long been regarded as the most efficient intrinsic lubricants in common use. Concentrations of two percent or less are usually effective.

Other traditional intrinsic lubricants include hydrogenated and partially hydrogenated vegetable oils, animal fats, polyethyleneglycol, polyoxyethylene monostearate, talc, light mineral oils, sodium benzoate, sodium lauryl sulphate, magnesium oxide and the like. See U.S. Pat. No. 3,042,531.

Lubricants, according to the present invention, can be used in amounts much greater than 1.5 weight percent, these amounts are much higher than those usually preferred, somewhere between about 0.25 and about 1.0 weight percent of the total composition.

Intrinsic lubricants pose certain serious difficulties when used in conventional tablets. Many lubricants materially retard the disintegration of tablets.

As used herein, a “hot melt binder” is one that is sufficiently rigid at standard ambient temperature and pressure but is capable of deformation or forming a semi-liquid state under elevated heat or pressure. Although the formulation of the invention need not contain a plasticizer to render it suitable for hot-melt granulation, plasticizers of the type described herein can be included.

Examples of hot-melt binders which can be used include acacia, tragacanth, gelatin, starch, cellulose materials such as methyl cellulose and sodium carboxy methyl cellulose, alginic acids and salts thereof, polyethylene glycol, guar gum, polysaccharide, bentonites, sugars, invert sugars, poloxamers (PLURONIC F68, PLURONIC F127), collagen, albumin, gelatin, cellulosics in nonaqueous solvents, and combinations of the above and the like.

Binders, for the hot-melt granulation, are preferably used in an amount of up to about 60 percent weight, more preferably up to about 40 percent weight and most preferably up to about 20 percent weight. A particularly preferred embodiment of the current invention comprises about 3 to about 8 percent weight of the total composition of a hot-melt binder. All binders used in this invention are suitable for hot-melt granulation. While the melting and/or softening point temperatures of these binders usually rise with increase of their molecular weights, preferable ones are those with a melting or softening point temperature less than about 150° C. However, binders having melting or softening points greater than about 150° C. can be used. Hot-melt binders having a melting or softening point temperature greater than about 150° C. may require use of a plasticizer during hot-melt granulation such that the binder melting or softening point temperature will be lowered below 150° C. Among the above-mentioned binders, polyethylene glycol is preferable, and that having a molecular weight of about 1000 to 8000 Da is more preferable.

The binder can be used in any form such as powder, granules, flakes or heat-molten liquid. While the amount of binder to be added can be modified, it is usually present in an amount less than about 10% by weight and preferably in the range of about 3-8% by weight of the granule.

When higher melting temperature, higher molecular weight or high softening temperature binders are employed, the hot-melt extrusion may require higher processing temperature, pressure and/or torque than when binders having a lower molecular weight, melting or softening temperature are employed. By including a plasticizer, and, optionally, an antioxidant, in a formulation, processing temperature, pressure and/or torque may be reduced. Plasticizers are not required in order to practice the invention. Their addition to the formulation is contemplated as being within the scope of the invention. Plasticizers are advantageously included in the granules when hot-melt extrudable binders having a melting or softening point temperature greater than 150° C. are employed.

As used herein, the term “plasticizer” includes all compounds capable of plasticizing the hot-melt extrudable binder of the invention. The plasticizer should be able to lower the melting temperature or glass transition temperature (softening point temperature) of the hot-melt extrudable binder. Plasticizers, such as low molecular weight PEG, generally broaden the average molecular weight of the hot-melt extrudable binder thereby lowering its glass transition temperature or softening point. Plasticizers also generally reduce the viscosity of a polymer melt thereby allowing for lower processing temperature and extruder torque during hot-melt extrusion.

Plasticizers useful in the invention can include, by way of example and without limitation, low molecular weight polymers, oligomers, copolymers, oils, small organic molecules, low molecular weight polyols having aliphatic hydroxyls, ester-type plasticizers, glycol ethers, poly(propylene glycol), multi-block polymers, single block polymers, low molecular weight poly(ethylene glycol), citrate ester-type plasticizers, triacetin, propylene glycol and glycerin.

Such plasticizers can also be ethylene glycol, 1,2-butylene glycol, 2,3-butylene glycol, styrene glycol, diethylene glycol, triethylene glycol, tetraethylene glycol and other poly(ethylene glycol) compounds, monopropylene glycol monoisopropyl ether, propylene glycol monoethyl ether, ethylene glycol monoethyl ether, diethylene glycol monoethyl ether, sorbitol lactate, ethyl lactate, butyl lactate, ethyl glycolate, dibutylsebacate, acetyltributylcitrate, triethyl citrate, acetyl triethyl citrate, tributyl citrate and allyl glycolate.

It is contemplated and within the scope of the invention, that a combination of plasticizers may be used in the present formulation. One advantageous combination is that comprised of poly(ethylene glycol) and low molecular weight poly(ethylene oxide). The PEG based plasticizers are available commercially or may be made by a variety of methods, such as disclosed in Poly(ethylene glycol) Chemistry: Biotechnical and Biomedical Applications (J. M. Harris, Ed.; Plenum Press, N.Y.) the teachings of which are hereby incorporated by reference.

Additional pharmaceutical excipients can be included in the lubricant granulate as required by the ultimate application of the granulate. For example, the lubricant granulate of the invention can optionally include various other excipients such as binders, diluents, disintegrants, etc. well-known to the art. Examples of optional diluent or binder materials include lactose, starches, sodium alginate, dicalcium phosphate hydrate, sugars, acacia, agar, calcium carrageenan, alginic acid, algin, agarose powder, microcrystalline cellulose, collagen, colloidal magnesium silicate, colloidal silicon dioxide, pectin, gelatin, calcium sulfate, ethyl cellulose, polyacrylates, etc.

The resulting granules of the invention can be included in formulations containing active ingredients and particularly pharmacologically active agents. As used herein, the term “active ingredient” means a therapeutic compound, a flavoring agent, a sweetening agent, a vitamin, cleansing agent and other such compounds for pharmaceutical, veterinary, horticultural, household, food, culinary, pesticidal, agricultural, cosmetic, herbicidal, industrial, cleansing, confectionery and flavoring applications. When the granules are formulated into tablets, such tablets can also contain coloring agents, lubricants and the like. The granules of the invention can be formulated in a variety of forms such as a tablet, capsule, suspension, reconstitutable powder and suppository.

When a formulation including the granules and a therapeutic compound is included in a pharmaceutical tablet, the tablet's size and shape can be adapted for direct oral administration to a patient, such as a human patient. The pharmaceutical tablet is substantially completely disintegrable upon exposure to water and/or saliva. The granule is present in an amount effective to aid in disintegration of the tablet when the tablet is placed in the mouth of a patient.

Disintegrants that can be included in the composition comprise starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin and tragacanth. Disintegrants can comprise up to about 20 weight percent and preferably between about 2 and about 10 percent of the total weight of the composition.

Coloring agents can include titanium dioxide, and dyes suitable for food such as those known as F.D. & C. dyes and natural coloring agents such as grape skin extract, beet red powder, beta-carotene, annato, carmine, turmeric, paprika, etc. The amount of coloring used can range from about 0.1 to about 3.5 weight percent of the total composition.

Flavors incorporated in the composition may be chosen from synthetic flavor oils and flavoring aromatics and/or natural oils, extracts from plants, leaves, flowers, fruits and so forth and combinations thereof. These may include cinnamon oil, oil of wintergreen, peppermint oils, clove oil, bay oil, anise oil, eucalyptus, thyme oil, cedar leave oil, oil of nutmeg, oil of sage, oil of bitter almonds and cassia oil. Also useful as flavors are vanilla, citrus oil, including lemon, orange, grape, lime and grapefruit, and fruit essences, including apple pear, peach, strawberry, raspberry, cherry, plum, pineapple, apricot and so forth. Flavors that have been found to be particularly useful include commercially available orange, grape, cherry and bubble gum flavors and mixtures thereof. The amount of flavoring may depend on a number of factors, including the organoleptic effect desired. Flavors may be present in an amount ranging from about 0.5 to about 3.0 by weight based upon the weight of the composition. Particularly preferred flavors are the grape and cherry flavors and citrus flavors such as orange.

Tablets according to this aspect of the present invention can be manufactured by well-known tableting procedures. In common tableting processes, material that is to be tableted is deposited into a cavity, and one or more punch members are then advanced into the cavity and brought into intimate contact with the material to be pressed, whereupon compressive force is applied. The material is thus forced into conformity with the shape of the punches and the cavity. Various tableting methods are well known to those skilled in the art and not detailed herein.

Materials to be incorporated in the tablets, other than the therapeutic compound can be pretreated to form granules that readily lend themselves to tableting. This process is known as granulation. As commonly defined, “granulation” is any process of size enlargement whereby small particles are gathered together into larger, permanent aggregates to yield a free-flowing composition having a consistency suitable for tableting. Such granulated compositions may have consistency similar to that of dry sand. Granulation may be accomplished by agitation in mixing equipment or by compaction, extrusion or globulation.

The therapeutic compound(s) contained within a formulation containing the granules of said invention can be formulated as its pharmaceutically acceptable salts. As used herein, “pharmaceutically acceptable derivatives” refer to salts, esters, hydrates or solvates of the disclosed compounds. In the case of pharmaceutically acceptable salts, the parent pharmacologically active compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfonic, sulfamic, phosphoric, nitric and the like; and the salts prepared from organic acids such as amino acids, acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, furnaic, toluenesulfonic, methanesulfonic, ethane disulfonic, oxalic, isethionic, and the like.

The pharmaceutically acceptable salts of the present invention can be synthesized from the parent therapeutic compound that contains a basic or acidic moiety by conventional chemical methods. Generally, such salts can be prepared by reacting the free acid or base forms of these compounds with a predetermined amount of the appropriate base or acid in water or in an organic solvent, or in a mixture of the two. Generally, nonaqueous media are preferred. Lists of suitable salts are found in Remington's Pharmaceutical Sciences, 17th ed., Mack Publishing Company, Easton, Pa., 1985, p. 1418, the disclosure of which is hereby incorporated by reference.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The amount of therapeutic compound incorporated in each tablet may be selected according to known principles of pharmacy. An effective amount of therapeutic compound is specifically contemplated. By the term effective amount, it is understood that, with respect to for example pharmaceuticals, a pharmaceutically effective amount is contemplated. A pharmaceutically effective amount is the amount or quantity of a drug or pharmaceutically active substance which is sufficient to elicit the required or desired therapeutic response, or in other words, the amount which is sufficient to elicit an appreciable biological response when administered to a patient.

A particular embodiment of the present invention relates to the preparation of pharmaceutical compositions containing bisphosphonic acids, especially of (1-hydroxy-3-(N-methyl-N-pentyl) aminopropylidene-1,1-bisphosphonic acid (ibandronic acid) or pharmaceutically acceptable salts thereof for the treatment of disorders characterized by pathologically increased bone resorption, especially for the treatment of osteoporosis. More specifically, the present invention is to a hot-melt granulation process to prepare pharmaceutical compositions, the pharmaceutical compositions themselves and the use of those pharmaceutical compositions for the treatment of disorders characterized by pathologically increased bone resorption, especially for the treatment of osteoporosis.

The following examples are given for the purpose of illustrating the present invention and should not be considered as limiting the scope or spirit of the invention in any way.

### EXAMPLES

### Example 1

The hot-melt granulation of Stearic Acid prior to creating the composition within this example and of the invention herein, was performed by the following process:

Granulation of Stearic Acid

Melt PEG 6000 using hot bath heated at 60-100 C., Stir continuously until
solution turn clear and no evidence of lumps
Add Stearic Acid and continue mixing until solution turns clear
Add Avicel PH102 Continue mixing until uniform dispersion is evident
Pour the dispersion onto a suitable stainless steel tray and let it air dry in
room temp approx 25-30 C.
Break the dried material into small chunks
Assemble Fitz Mill with Knives Forward and 0040 screen
Mill the material and collect into a suitable container

| | |
|---|---|
| Amt Req'd in | Quantity per Unit |

| Description | Grams | mg | % |
|---|---|---|---|
| PEG 6000 | 500.00 | 500.00 | 30.30 |
| Stearic Acid | 750.00 | 750.00 | 45.45 |
| Avicel PH 102 | 400.00 | 400.00 | 24.24 |

The granulated stearic acid was then used to prepare the following composition:

| | Amt Req'd in | Quantity per Unit | |
|---|---|---|---|
| Description | Grams | mg | % |
| Ibandronate Sodium | 337.50 | 168.75 | 37.50 |
| Monohydrate | | | |
| Lactose Monohydrate | 257.48 | 128.74 | 28.61 |
| Plasdone | 45.00 | 22.50 | 5.00 |
| Microcrystalline Cellulose | 120.00 | 60.00 | 13.33 |
| Crospovidone | 45.00 | 22.50 | 5.00 |
| Colloidal Silicion Dioxide | 9.00 | 4.50 | 1.00 |
| Stearic acid Granules(PEG 6000 + | 86.02 | 43.01 | 9.56 |
| stearic acid) | | | |
| Total Tablet Weight | 900.00 | 450.00 | 100.00 |
| (Core) | | | |

FILM COATING

| | | | |
|---|---|---|---|
| Opadry II White Y--30-18037 | 27.00 | 13.50 | 3.00 |
| Purified water, Usp | 127.29 | q.s. | |
| Total Final Tablet Weight | 927.00 | 463.50 | |

The process used to prepare the above composition was as follows:

A Separately pass Ibandronate Sodium Monohydrate IBA/0010406 and Lactose Monohydrate NF #315DC through #30 mesh and mix for 5 minutes.

B Plasdone S 630, Microcrystalline Cellulose NF (Avicel PH 200), and Crospovidone NF and mix from step A prescreened through a #30 mesh, sandwich Plasdone S 630, Microcrystalline Cellulose NF (Avicel PH 200), and Crospovidone NF between mix from step A and mix in suitable V-blender for 10 minutes.

C Colloidal Silicion Dioxide and Stearic Acid Granules (PEG 6000+stearic acid) prescreened through a #30 mesh and add to the mix from step B and blended for 5 minutes in a suitable V-blender.

D Compress on a suitable tablet press.

The resulting tablets were analyzed and it was found that there was no issues with respect to weight variation and there were no friability problems. Ten samples of tablets were tested for tablet hardness. The hardness measured ranged from 8.0 to 9.1 Kp, which was in good agreement with the target hardness of 8.4 Kp. These results are indicative of the fact that a high concentration of stearic acid can be used in a pharmaceutical composition, when the stearic acid is provided as a granulate according to the present invention.

### Example 2

The hot-melt granulation of Stearic Acid prior to creating the composition within this example and of the invention herein, was performed by the following process:

Granulation of Stearic Acid

Melt Stearic Acid using hot bath heated at 60-100 C., Stir continuously
until solution turns clear and no evidence of lumps
Obtain about 200 g of boiling water and add HPMC E5, continue mixing
with spatula until evidence of good dispersion
Add Avicel PH102 to the solution from step A, continue mixing until
dispersion is obtained
Add dispersion from Step B to Step C mix using spatula until the
dispersion is uniform
Pour the dispersion onto a suitable stainless steel tray and let it air dry in
room temp approx 25-30 C.
Break the dried material into small chunks
Assemble Fitz Mill with Knives Forward and 0040 screen

| | |
|---|---|
| Amt Req'd in | Quantity per Unit |

| Description | Grams | mg | % |
|---|---|---|---|
| HPMC E5 | 80.00 | 80.00 | 7.81 |
| Stearic Acid | 750.00 | 750.00 | 73.24 |
| Avicel PH 102 | 194.00 | 194.00 | 18.95 |

The encapsulated stearic acid is then used to prepare the following composition:

| | Amt Req'd in | Quantity per Unit | |
|---|---|---|---|
| Description | Grams | mg | % |
| Ibandronate Sodium | 337.50 | 168.75 | 37.50 |
| Monohydrate | | | |
| Lactose Monohydrate | 204.88 | 102.44 | 22.76 |
| Plasdone | 45.00 | 22.50 | 5.00 |
| Microcrystalline Cellulose | 120.00 | 60.00 | 13.33 |
| Crospovidone | 45.00 | 22.50 | 5.00 |
| Colloidal Silicion Dioxide | 9.00 | 4.50 | 1.00 |
| Stearic acid Granules(8% HPMC | 138.62 | 69.31 | 15.4 |
| E5 + stearic acid) | | | |
| Total Tablet Weight (Core) | 900.00 | 450.00 | 100.00 |

FILM COATING

| | | | |
|---|---|---|---|
| Opadry II White Y--30-18037 | 27.00 | 13.50 | 3.00 |
| Purified water, Usp | 127.29 | q.s. | |
| Total Final Tablet Weight | 927.00 | 463.50 | |

The process used to prepare the above composition was as follows:

A Separately pass Ibandronate Sodium Monohydrate IBA/0010406 and Lactose Monohydrate NF #315 DC through #30 mesh and mix for 5 minutes.

B Plasdone S 630, Microcrystalline Cellulose NF (Avicel PH 200), and Crospovidone NF and mix from step A prescreened through a #30 mesh, sandwich Plasdone S 630, Microcrystalline Cellulose NF (Avicel PH 200), and Crospovidone NF between mix from step A and mix in suitable V-blender 10 minutes.

C Colloidal Silicion Dioxide and Stearic Acid Granules (8% HPMC E5+Stearic Acid prescreened through a #30 mesh and add to the mix from step B and blended for 5 minutes in a suitable V-blender.

D Compress on a suitable tablet press.

The resulting tablets were analyzed and it was found that there was no issues with respect to weight variation and there were no friability problems. Ten samples of tablets were tested for tablet hardness. The hardness measured ranged from 6.5 to 7.2 Kp, which was comparable the target hardness of 7-8 Kp. These results are indicative of the fact that a high concentration of stearic acid can be used in a pharmaceutical composition, when the stearic acid is provided as a granulate according to the present invention.

All publications, patents and patent applications mentioned in this Specification are indicative of the level of skill of those skilled in the art to which this invention pertains and are herein incorporated by reference to the same extent as if each individual publication, patent, or patent applications was specifically and individually indicated to be incorporated by reference.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

1. A process for preparing a lubricant granulate comprising:
(a) preparing a molten mixture comprising a lubricant and a hot melt binder;
(b) allowing the molten mixture to cool; and
(c) milling the cooled mixture to form a granulate.

2. The process according to claim 1, wherein the hot melt binder comprises one or more of acacia, tragacanth, gelatin, starch, a cellulose ether, microcrystalline cellulose, sodium carboxy methyl cellulose, alginic acids and salts thereof, polyethylene glycol, guar gum, polysaccharide, bentonites, sugars, invert sugars, poloxamers, collagen, albumin, gelatin, cellulosics in nonaqueous solvents or a polyether glycol.

3. The process according to claim 2, wherein the hot melt binder is polyethylene glycol.

4. The process according to claim 2, wherein the hot melt binder is hydroxypropyl methylcellulose.

5. The process according to claim 1, wherein the lubricant is stearic acid, a metallic stearate, talc, colloidal silicon dioxide, hydrogenated or partially hydrogenated vegetable oils, corn starch, a polyethylene glycol, sodium benzoate, animal fat, polyoxyethylene monostearate, light mineral oil, sodium lauryl sulphate, magnesium oxide or sodium acetate.

6. The process according to claim 1, wherein the lubricant is stearic acid and the hot melt binder is polyethylene glycol.

7. The process according to claim 1, wherein the lubricant is stearic acid and the hot melt binder is hydroxypropyl methylcellulose.

8. The process according to claim 1, wherein one or more pharmaceutical binders, diluents or disintegrants are added to the molten mixture prior to cooling.

9. The process according to claim 8, wherein an aqueous dispersion of microcrystalline cellulose is added to the molten mixture prior to cooling.

10. A lubricant granulate prepared by the process of claim 1.

11. A lubricant granulate prepared by the process of claim 9, which comprises polyethylene glycol, stearic acid and microcrystalline cellulose.

12. The lubricant granulate according to claim 10, which comprises hydroxypropyl methylcellulose, stearic acid and microcrystalline cellulose.

13. The lubricant granulate according to claim 11, wherein the microcrystalline cellulose is Avicel PH 102.

14. A pharmaceutical composition comprising an active pharmaceutical ingredient (API) and a lubricant granulate according to claim 10.

15. The pharmaceutical composition according to claim 14, wherein the API is a bisphosphonic acid or a pharmaceutically acceptable salt thereof.

16. The pharmaceutical composition according to claim 15, wherein the bisphosphonic acid is ibandronic acid.

17. The pharmaceutical composition according to claim 15, wherein the API is sodium ibandronate.

18. The pharmaceutical composition of claim 14, wherein the amount of the lubricant is greater than 5% by weight of the total pharmaceutical composition.

19. The pharmaceutical composition of claim 14, wherein the amount of the lubricant is greater than 9% by weight of the total pharmaceutical composition.

20. The pharmaceutical composition of claim 14, wherein the amount of the lubricant is greater than 15% by weight of the total pharmaceutical composition.

21. The pharmaceutical composition of claim 14, wherein the composition is prepared as a solid oral pharmaceutical, the solid oral pharmaceutical being a tablet or a capsule.

22. A pharmaceutical composition that consists of Ibandronate sodium monohydrate, the lubricant granulate of claim 11, lactose monohydrate, plasdone, microcrystalline cellulose, Crospovidone and Colloidal Silicon Dioxide.

23. A pharmaceutical composition that consists of Ibandronate sodium monohydrate, the lubricant granulate of claim 12, lactose monohydrate, plasdone, microcrystalline cellulose, Crospovidone and Colloidal Silicon Dioxide.

24. A method for treating a disorder associated with increased bone resorption comprising administering to a patient the pharmaceutical composition of claim 15.

25. The method according to claim 24, wherein said disorder is osteoporosis, hypercalcaemia, tumour osteolysis or Paget's disease.

26. A lubricant granulate prepared by the process of claim 9.

27. The lubricant granulate according to claim 12, wherein the microcrystalline cellulose is Avicel PH 102.

28. A pharmaceutical composition comprising an active pharmaceutical ingredient (API) and a lubricant granulate according to claim 11.

29. A pharmaceutical composition comprising an active pharmaceutical ingredient (API) and a lubricant granulate according to claim 12.

30. A pharmaceutical composition comprising an active pharmaceutical ingredient (API) and a lubricant granulate according to claim 13.
